(19) United States
(12) Patent Application Publication
(10) Pub. No.: US 2008/0046023 A1
(43) Pub. Date: Feb. 21, 2008

(54) Electrode switch for a brain neuropacemaker

(76) Inventor: David R. Fischell, Fair Haven, NJ (US)

Correspondence Address: MORRISON & FOERSTER LLP, 755 PAGE MILL RD, PALO ALTO, CA, 94304-1018, US

(21) Appl. No.: 11/505,707

(22) Filed: Aug. 16, 2006

(51) Int. Cl. A61N 1/00

(52) U.S. Cl. 607/45

(57) ABSTRACT

Described here are systems for connecting a multiplicity of brain electrodes to an electronic medical device. In some variations, the system comprises at least two leads, where each lead has a conducting means connected to at least one electrode, a reconfigurable electrode switch with control logic attachable to the at least two leads, an electronics control module designed for therapeutic treatment of neurological disorders, and at least one multi-wire connecting cable to connect the electrode switch to the electronics control module. In some variations, the electrode switch is designed to allow transmission of electrical signals between any one of the at least two electrode leads and the control module. In other variations, the electrode switch is designed to allow transmission of signals from any combination of two or more of the electrodes through the connecting cable to the control module.

### FIELD OF USE

This invention is in the field of devices to treat neurological diseases that originate in the brain.

### BACKGROUND OF THE INVENTION

There are several neurological diseases that are characterized by certain electrical discharges that can permeate throughout the brain causing certain human dysfunctions such as epileptic seizures or Parkinson's tremors.

Deep brain stimulation systems like the Medtronic Activa used for treating Parkinson's tremor are typically implanted in the chest with electrode leads tunneled up the neck to the head. This is limiting if multiple stimulation sites are desired as it requires that all accessible electrodes be tunneled from the head through the neck to the implant location.

In U.S. Pat. No. 6,427,086 by Fischell et al (which is included herein by reference) there is described an intra-cranially implantable responsive neurostimulator system that uses electrical stimulation for the treatment of neurological diseases such as epilepsy or Parkinson's. However, the Fischell et al application describes direct connection of intracranial electrodes to the responsive neurostimulator which is inherently limited as to the total number of electrodes that can be accessed by the responsive neurostimulator. Ideally, one would prefer to implant many multi-electrode leads where a subset of the electrodes can be accessed at any time by the responsive neurostimulator.

### SUMMARY OF THE INVENTION

The present invention is a reconfigurable electrode switch that allows a large number of multi-electrode leads to be cross connected to the existing inputs of a brain neuropacemaker. The electrode switch can be designed to reconfigure the electrodes that can be accessed by the neuropacemaker on a lead switching or electrode switching basis. In the lead switching embodiment, all the electrodes on a selected lead are switched through to the neuropacemaker. In the electrode switching embodiment, any electrode on any lead can be switched through to the neuropacemaker.

Reconfiguration of the electrode switch can be accomplished in one of three ways:
1. from commands transmitted by the neuropacemaker through a separate wired data control lead that physically connects to a feed through in the case of the neuropacemaker.
2. by multiplexing the control signals onto the electrode signal wires connecting the electrode switch to the neuropacemaker or
3. by wireless data communication, including a subcutaneous communication coil or antenna that can communicate with the telemetry capability of a electrode switch programmer.

It is envisioned that the electrode switch can either be self powered with its own battery, externally powered by magnetic induction during programming or it can be powered from the neuropacemaker via the control wires, electrode signal wires or separate power wires. Ideally, the electrode switch requires power only during reconfiguration and will maintain its configuration without needing to be powered.

The electrode switch may be either an analog switch or a digital switch where the input signals from the electrodes are first converted to digital signals and the switching uses digital switching techniques such as time division multiplexing. The analog switch is the preferred embodiment as it can be constructed to not require power except during reconfiguration.

It is further envisioned that while the preferred embodiment electrode switch connects to electrodes placed in the vicinity of the patient's brain, the present invention electrode switch is applicable to electrodes implanted elsewhere for the purpose of sensing electrical signals from the patient's body.

Thus it is an object of this invention to have an electrode switch that can increase the number of brain electrodes accessible by a brain neuropacemaker.

Another object of the present invention is to have an electrode switch that acts as a lead switch by allowing selection and reconfiguration of the leads accessible by an implanted device.

Another object of the present invention is to have an electrode switch that acts as an electrode switch by allowing selection and reconfiguration of the individual electrodes accessible by an implanted device.

Another object of the present invention is to have the electrode switch controlled by the processor of the brain pacemaker.

Still another object of the present invention is to have the electrode switch be an analog crossbar switch.

Still another object of the present invention is to have an electrode switch constructed using MEMS technology.

Still another object of the present invention is to have the electrode switch powered by any of: the neuropacemaker battery, a self contained battery or an external power source using magnetic induction.

Still another object of the present invention is to have the electrode switch powered by external equipment during programming after which it locks into the programmed configuration which it maintains without the need for power.

Yet another object of the present invention is to have the electrode switch communicate with the neuropacemaker over a wire.

Yet another object of the present invention is to have the electrode switch communicate with the neuropacemaker over a wireless connection.

Yet another object of the present invention is to have the electrode switch communicate with equipment external to the patient's body.

These and other objects and advantages of this invention will become obvious to a person of ordinary skill in this art upon reading of the detailed description of this invention including the associated drawings.

### BRIEF DESCRIPTION OF THE DRAWINGS

FIG. 1 is a sketch of the prior art cranially implanted neuropacemaker.

FIG. 2 is a sketch of the prior art pectorally implanted neuropacemaker.

FIG. 3 is a sketch of the present invention electrode switch used with a cranially implanted neuropacemaker.

FIG. 4 is a sketch of the present invention electrode switch used with a pectorally implanted neuropacemaker.

FIG. 5 is a block diagram of a first embodiment of an implanted system having a lead switching electrode switch programmed from the implanted neuropacemaker.

FIG. 6 is a block diagram of a second embodiment of an implanted system having a lead switching electrode switch programmed from an external programmer.

FIG. 7 is a block diagram of a third embodiment of an implanted system having a electrode switching electrode switch.

FIG. 8 is a block diagram of an example of the electrode switch of FIG. 7.

### DETAILED DESCRIPTION OF THE DRAWINGS

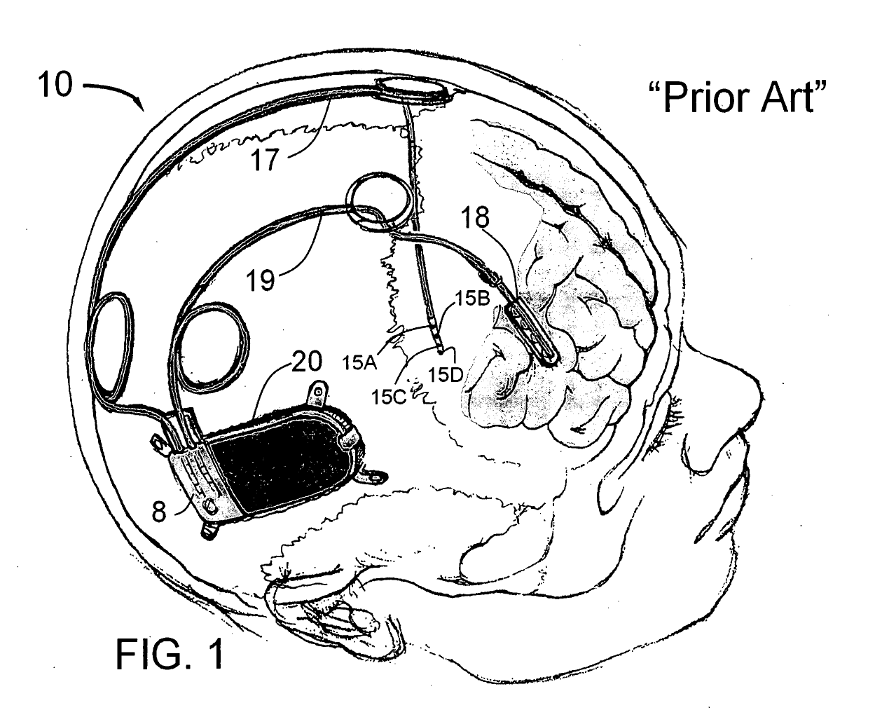

FIG. 1 illustrates the configuration of a prior art cranially implanted system 10 for the treatment of neurological disorders as it would be situated under the scalp of a human head having a control module 20, depth electrodes 15A, 15B, 15C & 15D with multi-wire lead 17 connected through the connector 8 to the control module 20. It is envisioned that the control module 20 is permanently implanted into the side of the skull in a location where the skull is fairly thick. The electrodes 15A, 15B, 15C and 15D would be located in an internal structure of the patient's brain such as the thalamus, hippocampus or cerebellum. The brain surface electrode array 18 with connecting multi-wire lead 19 would be placed under the cranium and below the dura mater. The lead 19 would connect to the control module 20 through the connector 8. The electrode array 18 is a line array with four electrodes placed in a single row with spacing of at least ½ centimeter. Although depth electrodes with anywhere from 1 to 20 electrodes and surface electrode arrays with up to 64 electrodes are envisioned it is difficult to connect more than two leads with eight electrodes at a time. The typical embodiment, however is depth and linear arrays each with 4 electrodes spaced by either ½ or 1 centimeter.

It is advantageous to implant additional leads during the system implantation procedure to be in place if additional brain locations need to be accessed at a future time. Unfortunately, if more leads are implanted, then surgery is required to switch from one lead to another. Furthermore, although the control module 20 might be able to process signals from eight separate channels, the two lead limitation restricts the ability to use eight electrodes that are located in more than two sites.

Throughout the detailed description of the present invention, the terminology “the electrodes 15A through 15N” is meant to include all electrodes 15A, 15B, 15C, . . . to 15N inclusive where N may be any integer between 1 and 500. Similar terminology using the words “through” or “to” for other groups of objects (i.e., wires 17A through 17N) will have a similar inclusive meaning.

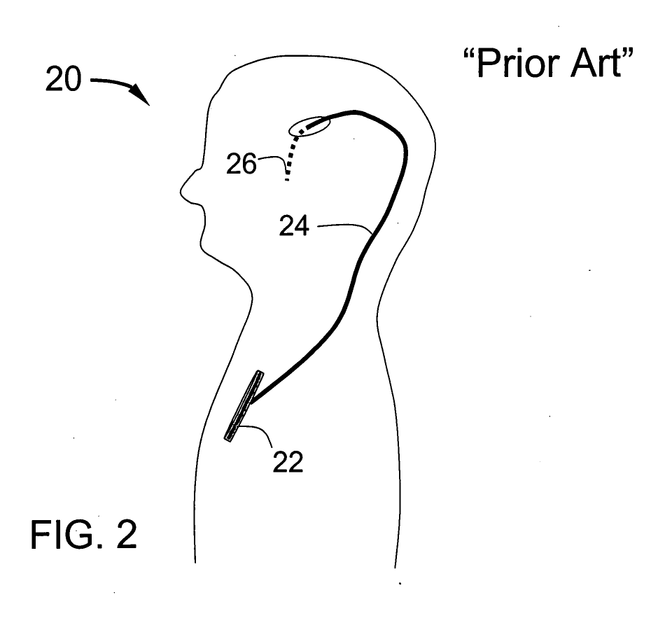

FIG. 2 illustrates the configuration of a prior art pectorally implanted system 20 for the treatment of neurological disorders as it includes a control module 22 implanted under the skin of the chest or abdomen and a lead 24 terminating in an electrode array 26. The “Activa” pacemaker from Medtronic is such a device which attaches to a single lead and is currently implanted for the treatment of Parkinson's disease. It is also difficult to use a pectorally implanted neuropacemaker with a multiplicity of brain electrode arrays as multiple leads might need to be tunneled subcutaneously down the neck to the implant site.

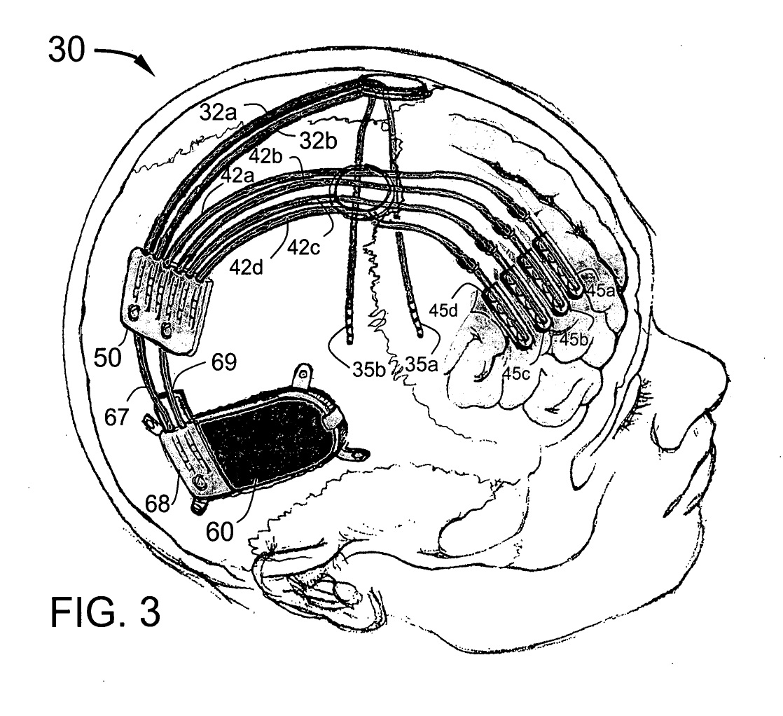

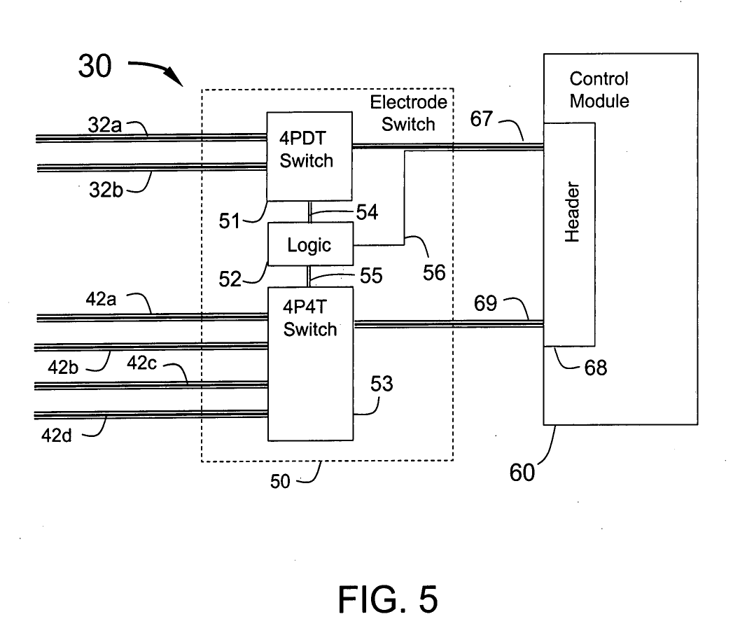

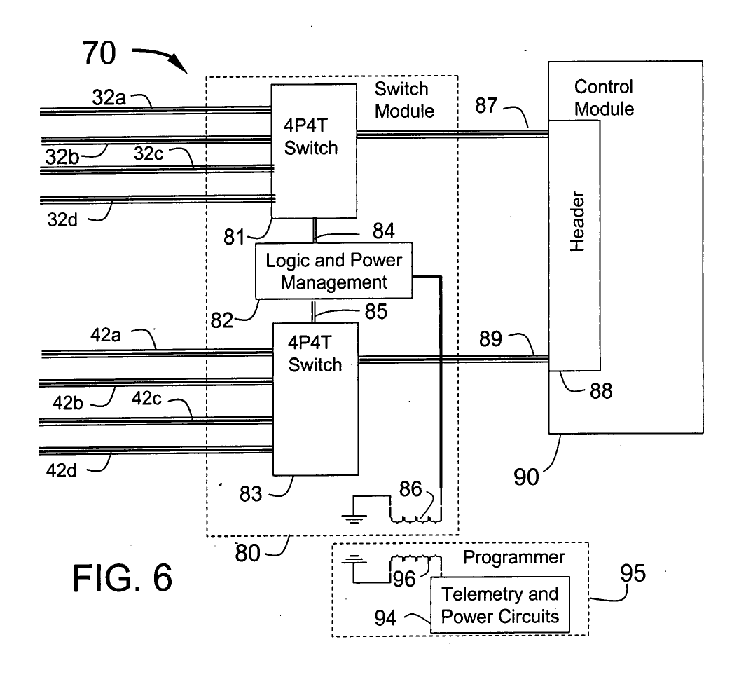

FIG. 3 illustrates the configuration of the present invention cranially implanted system for the treatment of neurological disorders 30 having a control module 60, two depth electrode arrays 35a and 35b with leads 32a and 32b, four surface electrode arrays 45a through 45d respectively having leads 42a through 42d respectively. In FIG. 3 each lead 32a, 32b, and 42a through 42d is shown having four electrodes. The six leads therefore contain a total of 24 electrodes. The six leads shown in FIG. 3 also connect into the electrode switch 50. Two interconnect cables 67 and 69 connect the electrode switch 50 to the control module 60 through the header 68. There are embodiments envisioned in which the electrode switch 50 would be used to flexibly connect the electrodes 35a, 35b, and 45a through 45d to the control module 60. The first embodiment uses lead switching to change which two of the six leads are connected to the control module through the cables 67 and 69. The control module can only access the electrodes on two leads (e.g. one depth array and one surface array) at any one time. The advantage of this embodiment is that it is relatively simple to implement using two reconfigurable four pole multi-position switches as will be seen in FIGS. 5 and 6.

A more desirable embodiment of the present invention implements the equivalent of a crossbar switch used in telecommunications allowing electrode switching to connect any electrode from any of the lead to the control module 60. For example, if the control module is designed to access eight channels then any eight of the 24 electrodes shown could be accessed by the control module 60 over the cables 67 and 69. Such a crossbar switch could be constructed from eight single pole 24 position switches. Innovations in MEMS technology which applies semiconductor etching methods to small mechanical devices can be used to make such a switch small enough to be practical for the implanted system 30.

Although the system 30 of FIG. 3 shows 24 electrodes in 6 leads, it is envisioned that the electrode switch 50 could be configured for as many as 16 leads each having as many as 64 electrodes. The arrays can be any combination of surface or depth electrode arrays or for that matter any type of electrode configuration for collecting signals and/or electrical stimulation of portions of the human body.

It is envisioned that the electrode switch 50 can either be self powered with its own battery, externally powered by magnetic induction during programming or it can be powered from the neuropacemaker via the control wires, electrode signal wires or separate power wires. Ideally, the electrode switch requires power only during reconfiguration and will maintain its configuration without needing to be powered.

The electrode switch 50 may be either an analog switch or a digital switch where the input signals from the electrodes are first converted to digital signals and the switching uses digital switching techniques such as time division multiplexing. The preferred embodiment is an analog switch which is shown in FIGS. 5,6,7 and 8.

It is further envisioned that while the preferred embodiment electrode switch 50 connects to electrodes placed in the vicinity of the patient's brain, the present invention electrode switch is applicable to electrodes implanted elsewhere for the purpose of electrical stimulation and/or sensing electrical signals from the patient's body.

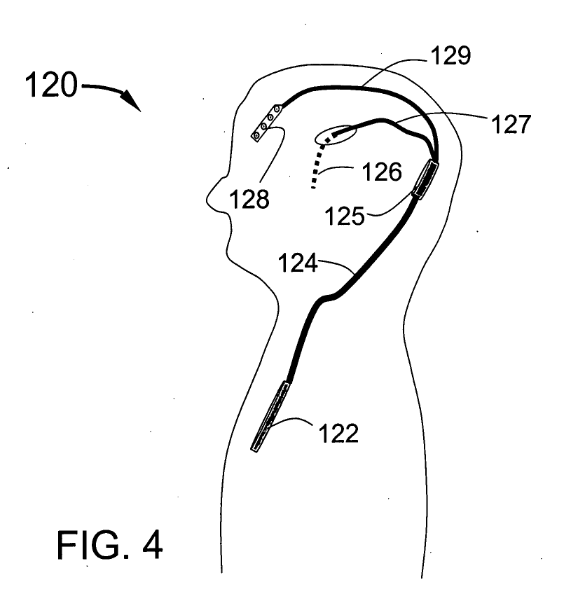

FIG. 4 illustrates the configuration of the present invention pectorally implanted system 120 for the treatment of neurological disorders as it includes a control module 122 implanted under the skin of the chest or abdomen and a connecting cable 124 that connects the control module 122 to the electrode switch 125. FIG. 4 shows a depth electrode array 126 with lead 127 and a surface electrode array 128 with lead 129 both connecting into the electrode switch 125. This technique could also be used with more than two electrode arrays including any combination of depth and surface electrodes. This embodiment would be extremely practical as the current use of the “Activa” pacemaker from Medtronic to treat bilateral Parkinson's disease requires two separate pacemaker implants and this technique could allow two sites to be stimulated by a single pacemaker.

FIG. 5 is a block diagram of a first embodiment of the system 30 having a lead switching electrode switch 50. The depth electrode leads 32a and 32b connect into a 4PDT switch 51 controlled by the logic circuitry 52 that allows either lead 32a or 32b to be switched through to the multi-wire cable 67 connecting to the header 68 on the control module 60. All four electrodes on the switched lead connect through and there is no ability in this embodiment to access electrodes from more than one lead.

The surface electrode leads 42a through 42d connect into a 4P4T switch 53 controlled by the logic circuitry 52 that allows any one of the leads 42a through 42d to be switched through to the multi-wire cable 69 connecting to the header 68 on the control module 60. All four electrodes on the switched lead connect through to the cable 69 and there is no ability in this embodiment to access electrodes from more than one lead.

Although four pole switches 51 and 52 are shown here in FIG. 5, it is envisioned that switches with less or more poles could be used. For example if the leads each have eight electrodes instead of four, the eight pole switches would be used.

It is also envisioned that the electrode switch 50 could be configured to select a specific row, column or sub-group of electrodes in a two dimensional electrode grid array such as those used in brain mapping procedures prior to epilepsy surgery.

In the embodiment of the electrode switch 50 shown in FIG. 5, the logic 52 that controls the switching of the incoming leads is controlled by signals sent from the control module 68 through the control channel 56. The control channel is typically a wire or wires that can be part of or separate from the connecting cables 67 and 69. It is also envisioned that the control signals can be multiplexed onto one or more of the wires in the multi-cables 67 or 69.

In this embodiment the logic circuitry 52 can be powered from the control module 68 as needed. Programming the electrode switch 50 in this embodiment would be accomplished through the telemetry and command capabilities of the control module 60 using a programmer (not shown) as described by Fischell et al in U.S. Pat. No. 6,016,449 which is included herein by reference.

FIG. 6 is a block diagram of a second embodiment of the system 70 having a lead switching electrode switch 80. The depth electrode leads 32a through 32d connect into a 4P4T switch 81 controlled by the logic and power management circuitry 82 that allows any one of the leads 32a through 32d to be switched through to the cable 87 connecting to the header 88 on the control module 90. All four electrodes on the switched lead connect through the 4P4T switch 81 and there is no ability in this embodiment to access electrodes from more than one of the leads 32a through 32d.

The surface electrode leads 42a through 42d connect into a 4P4T switch 83 controlled by the logic and power management circuitry 82 that allows any one of the leads 42a through 42d to be switched through to the cable 89 connecting to the header 88 on the control module 90. All four electrodes on the switched lead connect through the 4P4T switch 83 and there is no ability in this embodiment to access electrodes from more than one leads 42a through 42d.

Although four pole switches 81 and 82 are shown here in FIG. 6, it is envisioned that switches with less or more poles could be used. For example if the leads 42a through 42d have eight electrodes instead of four, the eight pole switches would be used. Although 4 throw switches 81 and 82 are shown here in FIG. 6, a larger number of throws is also envisioned. For example, if the switch 81 were to be used with 8 leads each with 8 electrodes, rather than the 4 leads with 4 electrodes shown then an 8P8T (eight pole, eight throw) switch would be needed.

In the embodiment of the electrode switch 50 shown in FIG. 5, the logic 52 that controls the switching of the incoming leads is controlled by signals sent from the control module 68 through the control channel 56. In the electrode switch 80 of FIG. 6, programming signals are sent directly from a switch programmer 95 through a coil antenna 86 to the logic and power management circuitry 82.

In this embodiment the logic and power management circuitry 82 can either be self powered from a small internal battery or it can be powered as needed by the programmer through current induction through the skin using the programmer coil 96 in close proximity to the electrode switch coil 86. Ideally, the switches 81 and 82 do not require power once they are configured and power would only be needed to change the configuration.

The embodiment of FIG. 6 has the advantage that it can be used with existing implantable devices that do not have the capability to control an electrode switch.

In either the embodiments of the systems 30 and 70 of FIGS. 5 and 6 the incoming leads need not be segregated into two groups where the depth electrode leads attach to one switch (51 or 81) and the surface electrode leads to the other but the leads can be mixed in any combination as chosen by the patient's physician at the time of implant.

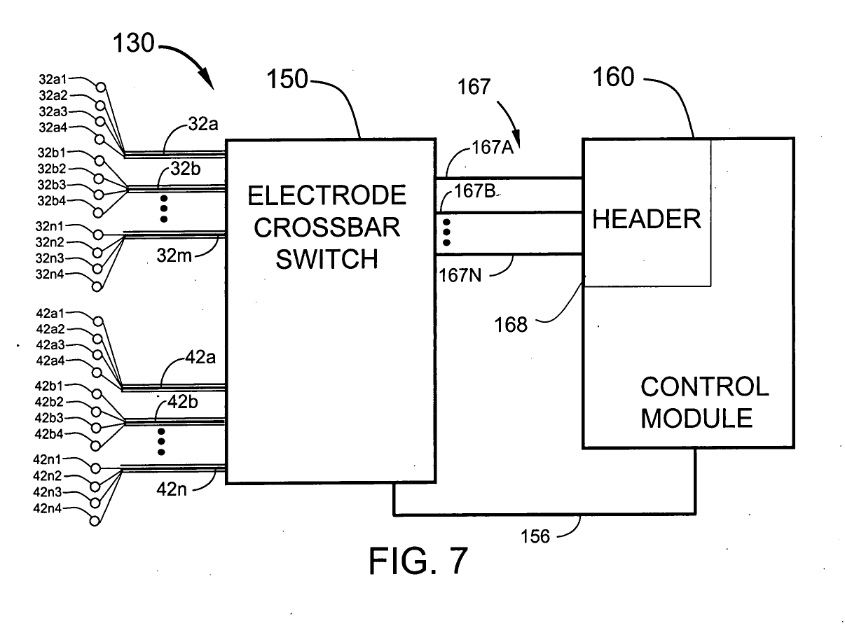

FIG. 7 shows a block diagram of the system 130 having an electrode switching embodiment of the present invention electrode switch 150. The electrode switch 150 which performs electrode switching is typically called a “crossbar” switch. A total of m depth electrode leads 32a through 32m each have 4 electrodes 32a1 through 32a4, 32b1 through 32b4 and so on. A total of n surface electrode leads 42a through 42n each have 4 electrodes 42a1 through 42a4, 42b1 through 42b4 and so on. Each electrode connects to a single conductor in the leads 32a through 34m and 42a through 42n. These conductors attach to the m+n inputs of the crossbar switch 150. The outputs of the crossbar switch 150 are “N” conductors 167A through 167N in a cable 167 that connects to the header 168 of the control module 160. N, m and n can all be different values. In this embodiment any N of the m+n total electrodes can be accessed by the control module 160.

For example if there are 4 depth electrode leads (m=4) and 4 surface electrode leads (n=4) each with 4 electrodes and the cable 167 has eight conductors (N=8) then any eight of the total of 32 electrodes can be simultaneously accessed by the control module 160.

The system 130 includes a control channel 156 between the control module 160 and the crossbar switch 150 similar to the control channel 56 of FIG. 5. It is also envisioned that the electrode crossbar switch 150 could be self-powered by a small battery or externally powered and controlled similar to the electrode switch 80 of FIG. 6.

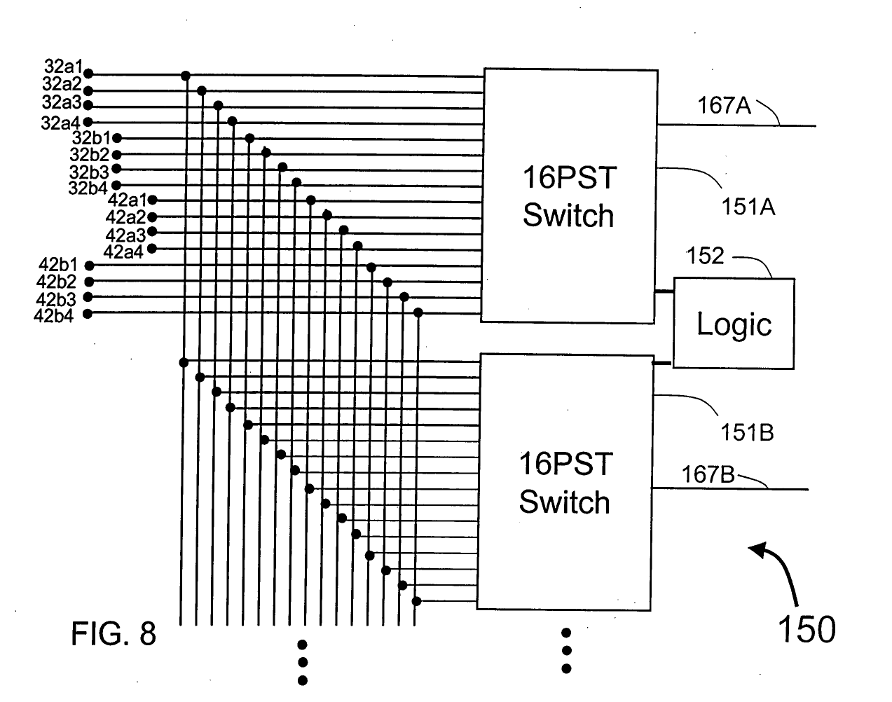

FIG. 8 is a block diagram of an example of the crossbar switch 150 of FIG. 7. In this example, there are 16 electrodes that may be accessed by the crossbar switch 150. These are eight depth electrodes 32a1 through 32a4 and 32b1 through 32b4 and eight surface electrodes 42a1 through 42a4 and 42b1 through 42b4. The crossbar switch 150 includes N 16 pole single throw switches 151A, 151B through 151N. The conducting wire from each of the 16 electrodes, is connected in turn to each of the 16 PST switches 151A through 151N. The 16 PST switches 151A, 151B though 151N are controlled by the logic circuitry 152. In this embodiment any one of the output conductors 167A, 167B through 167N corresponding to the switches 151A, 151B through 151N can be connected through to any one of the 16 electrodes. In this way, this crossbar switch embodiment has the flexibility to allow any combination of input electrodes to connect in any desired configuration to input channels of the control module of FIG. 7.

Any of the electrode switch embodiments can be applicable whether the control module is implanted in the cranial bone, the chest, the abdomen or any other subcutaneous location within the human body. It is also envisioned that a similar system could be used to make a multiplicity of implanted electrodes accessible to an external control module outside of the patient's body. Various other modifications, adaptations, and alternative designs are of course possible in light of the above teachings. Therefore, it should be understood at this time that within the scope of the appended claims the invention may be practiced otherwise than as specifically described herein.

What is claimed is:

1. A system for connecting a multiplicity of brain electrodes to an electronic medical device including:
at least two leads, each lead having conducting means connected to at least one electrode;
a reconfigurable electrode switch with control logic attachable to the at least two leads;
an electronics control module designed for therapeutic treatment of neurological disorders of a human patients; and,
at least one multi-wire connecting cable to connect the electrode switch to the electronics control module, the electrode switch being designed to allow transmission of electrical signals between any one of the at least two electrode leads and the control module.

2. The system of claim 1 where at least one of the leads is a depth electrode lead.

3. The system of claim 1 where at least one of the leads is a surface electrode lead.

4. The system of claim 3 where the surface electrode lead is a two dimensional grid array of electrodes.

5. The system of claim 4 where the electrode switch is designed to allow transmission of electrical signals between any sub-group of electrodes of the two dimensional grid array of electrodes and the control module electrodes.

6. The system of claim 4 where the electrode switch is designed to allow transmission of electrical signals between any single row of the two dimensional grid array of electrodes and the control module.

7. The system of claim 4 where the electrode switch is designed to allow transmission of electrical signals between any single column of the two dimensional grid array of electrodes and the control module.

8. The system of claim 1 where the connecting cable includes a control channel, the control module being designed to send signals over the control channel to the control logic of the electrode switch, the control signals being designed to set the configuration of the electrode switch.

9. The system of claim 1 where the connecting cable includes a power channel, the power channel being designed to allow the circuitry of the electrode switch to be powered from the control module.

10. The system of claim 1 where the electrode switch is self powered using a battery.

11. The system of claim 1 where the electrode switch is externally powered during programming.

12. The system of claim 1 where the electrode switch will maintain its configuration without the need for electrical power.

13. The system of claim 1 where the electronics control module is implanted under the patient's scalp.

14. The system of claim 1 where the electronics control module is implanted in the patient's chest.

15. The system of claim 1 where the electronics control module is implanted in the patient's abdomen.

16. A system for connecting a multiplicity of brain electrodes to an electronic medical device including
at least two leads, each lead having conducting means connected to at least one electrode;
a reconfigurable electrode switch with control logic attachable to the at least two leads;
an electronics control module designed for therapeutic treatment of neurological disorders of a human patients; and,
at least one multi-wire connecting cable to connect the electrode switch to the electronics control module, the electrode switch being designed to allow transmission of signals from any combination of two or more of the electrodes through the connecting cable to the control module.

17. The system of claim 16 where at least one of the leads is a depth electrode lead.

18. The system of claim 16 where at least one of the leads is a surface electrode lead.

19. The system of claim 18 where the surface electrode lead is a two dimensional grid array of electrodes.

20. The system of claim 19 where the electrode switch is designed to allow transmission of electrical signals between any sub-group of electrodes of the two dimensional grid array of electrodes and the control module.

21. The system of claim 19 where the electrode switch is designed to allow transmission of electrical signals between any single electrode of the two dimensional grid array of electrodes and the control module.

22. The system of claim 16 where the connecting cable includes a control channel, the control module being designed to send signals over the control channel to the control logic of the electrode switch, the control signals being designed to set the configuration of the electrode switch.

23. The system of claim 16 where the connecting cable includes a power channel, the power channel being designed to allow the circuitry of the electrode switch to be powered from the control module.

24. The system of claim 16 where the electrode switch is self powered using a battery.

25. The system of claim 16 where the electrode switch is externally powered during programming.

26. The system of claim 16 where the electrode switch will maintain its configuration without the need for electrical power.

27. The system of claim 16 where the electronics control module is implanted under the patient's scalp.

28. The system of claim 16 where the electronics control module is implanted in the patient's chest.

29. The system of claim 16 where the electronics control module is implanted in the patient's abdomen.
